(19) United States
(12) Patent Application Publication
(10) Pub. No.: US 2007/0299260 A1
(43) Pub. Date: Dec. 27, 2007

(54) Method for Preparing Hexahydro-8-Hydroxy-2, 6-Methano-2H-Chinolizin-3 (4H) -One Esters

(75) Inventors: Hans-Ulrich Bichsel, Hörhausen (CH); Vit Lellek, Zurich (CH)

Correspondence Address: HAMMER & HANF, PC, 3125 SPRINGBANK LANE, SUITE G, CHARLOTTE, NC, 28226, US

(73) Assignee: CILAG LTD., Schaffhausen (CH)

(21) Appl. No.: 11/719,968

(22) PCT Filed: Nov. 25, 2004
(86) PCT No.: PCT/CH04/00708

§ 371 Date: Jun. 6, 2007

(51) Int. Cl. C07D 451/00

(52) U.S. Cl. 546/94

(57) ABSTRACT

The invention concerns a method for preparing optionally substituted 3-indolcarboxylic acid esters, with hexahydro-8-hydroxy-2,6-methano-2H-chinolizin-3(4H)-one. The invention is characterized in that the optionally substituted 3-indolcarboxylic acid is converted by means of a suitable halogenating agent, into corresponding acid halide, preferably corresponding acid chloride, and the latter is transformed with hexahydro-8-hydroxy-2,6-methano-2H-chinolizin-3(4H)-one. The invention is characterized in that the entire reaction occurs in acid medium with a maximum pH of 7.

The present invention relates to a process for preparing esters of 3-indolecarboxylic acid with hexahydro-8-hydroxy-2,6-methano-2H-quinolizin-3(4H)-one, especially dolasetron. The compound dolasetron is known per se and corresponds to the chemical name trans-8-(3-indolylcarbonyloxy)hexahydro-2,6-methano-2H-quinolizin-3(4H)-one.

EP 0 266 730 describes compounds of the dolasetron type, and processes for their preparation are also disclosed. What is proposed is the conversion of 3-indolecarboxylic acid to the corresponding acid chloride and then reaction with the alcohol or an alkali metal salt of the alcohol (i.e. the quinolizine compound). However, it has been found that the yield in the process proposed is low and the reaction proceeds slowly and incompletely, and numerous by-products additionally form. This is also the case even when the reaction, as described in EP 0 266 730, is performed in the presence of a heavy metal salt, for example of a silver salt.

It has now been found that the reaction proceeds with significantly improved yield and without the formation of by-products which are difficult to remove when the entire reaction is performed in acidic medium at an acid value (pH) of at most 7 (pH≦7). This means that the 3-indolecarboxylic acid is converted in acidic medium to the corresponding acid chloride, and the resulting acid chloride is reacted with the alcohol, i.e. with the quinolizine compound, likewise in acidic medium. In this way, it is not necessary to use an alkali metal salt of the alcohol or a heavy metal salt. The inventive reaction proceeds rapidly and with good yield, for example with a yield of about 80% at a purity of >99.5% without the formation of by-products which are difficult to remove.

The present invention is defined in the claims. In particular, the present invention relates to a process for preparing optionally substituted esters of optionally substituted 3-indolecarboxylic acid with hexahydro-8-hydroxy-2,6-methano-2H-quinolizin-3(4H)-one, especially of dolasetron, by converting optionally substituted 3-indolecarboxylic acid with a suitable halogenating agent to the corresponding acid halide, preferably to the acid chloride, and reacting the latter with hexahydro-8-hydroxy-2,6-methano-2H-quinolizin-3(4H)-one, characterized in that the overall reaction is performed in an acidic medium at an acid value (pH) of not more than 7. Subsequently, the ester formed can be released by adding base and optionally converted to a salt. Preference is given to the reaction of the acid halide with endo-hexahydro-8-hydroxy-2,6-methano-2H-quinolizin-3(4H)-one.

The entire reaction is performed in acidic medium, preferably at an acid value (pH) of at most 6.5, preferably at an acid value of at most 6. For the establishment of an acidic pH in the reaction mixture, preference is given to using a very strong acid, preferably an inorganic acid, preferably sulfuric acid, and/or an organic acid, preferably methanesulfonic acid, benzenesulfonic acid, toluenesulfonic acid, trifluoromethanesulfonic acid and/or camphorsulfonic acid, preferably sulfuric acid, methanesulfonic acid, benzenesulfonic acid, toluenesulfonic acid and/or trifluoromethanesulfonic acid, preferably methanesulfonic acid.

A particular embodiment consists in using all three components in the reaction in the same reaction apparatus, i.e. converting the 3-indolecarboxylic acid in acidic medium in the apparatus to the corresponding acid halide, and then adding the quinolizine compound to the reaction mixture. However, it is also possible to initially charge the 3-indolecarboxylic acid in acidic medium together with the alcohol, i.e. the quinolizine compound, and to prepare the corresponding acid halide in situ by adding a suitable halogenating agent, said acid halide then being reacted further in the reaction mixture with the quinolizine compound.

A further preferred embodiment consists in preparing a salt of the quinolizine compound, i.e. of hexahydro-8-hydroxy-2,6-methano-2H-quinolizin-3(4H)-one, with a very strong acid beforehand, for example the salt formed with sulfuric acid, such as the hydrogensulfate, or the salt of an organic sulfonic acid, such as the salt with methylsulfonic acid or with toluenesulfonic acid, and using this salt in the reaction. When such an acidic salt is used in the reaction, the acid value is stabilized by the acid, in accordance with the invention, within the acidic range during the overall reaction without any need to add further acid to the reaction mixture. In addition, a very pure quinolizine compound can be introduced into the reaction as a starting material, since the salt can be prepared in very high purity, for example by crystallization. When, for example, the mesylate is used, the reaction can be formulated according to scheme 1, as follows:

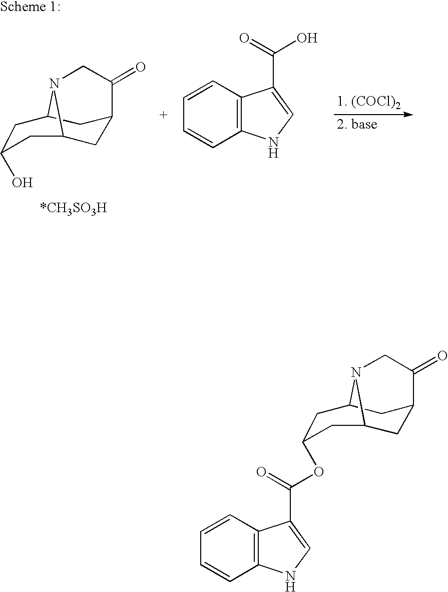

A further preferred embodiment consists in preparing a salt of 3-indolecarboxylic acid with a very strong acid beforehand, for example the salt formed with sulfuric acid, such as the hydrogensulfate, and using this salt in the reaction. When such an acidic salt is introduced into the reaction, the acid value is stabilized by the acid, in accordance with the invention, within the acidic range during the overall reaction without any need to add further acid to the reaction mixture. In addition, a very pure 3-indolecarboxylic acid can be introduced into the reaction as a starting material, since the salt can be prepared in very high purity, for example by crystallization. The salt can subsequently be converted to the carbonyl halide and reacted with the hydroxyl group of the quinolizine compound.

In this case, the reaction mixture consisting of the sulfate or sulfonate of the alcohol, the acid halide and any halogenating agent still present can be heated during the reaction up to reflux temperature of the solvent used (e.g. 2-butanone) without by-products occurring. The reaction is extremely short at 1-2 hours at elevated temperature. It is surprising that the inventive reaction, especially with the endo-alcohol, succeeds so efficiently through use of the sulfate or of a sulfonate.

Analogously, the reaction mixture consisting of the sulfate or sulfonate of 3-indolecarboxylic acid and the acid halide and any halogenating agent still present and also of the quinolizine compound can be heated during the reaction up to reflux temperature of the solvent used (e.g. 2-butanone) without the occurrence of amounts of by-products which reduce the yield. The reaction time at 1-2 hours at elevated temperature is likewise very short. The quinolizine compound can be used as a free base or as a salt, as described above.

When the quinolizine compound, preferably the endo-alcohol, is used as the salt of a strong acid, this is preferably the sulfate (salt of sulfuric acid), preferably as the hydrogensulfate, or the salt of an organic sulfonic acid, preferably the mesylate (salt with methylsulfonic acid), the besylate (salt with benzenesulfonic acid), the tosylate (salt with toluene-sulfonic acid), the trifluoromethanesulfonate, or the camphorsulfonic acid salt, preferably the hydrogen-sulfate, the mesylate, the besylate, the tosylate or the trifluoromethanesulfonate, preferably the mesylate.

Preference is given to using a corresponding salt of the quinolizine compound, preferably of the endo-alcohol, and reacting with the acid halide, preferably the acid chloride, of the 3-endolecarboxylic acid.

Examples of suitable halogenating agents are compounds known per se, such as oxalyl chloride, thionyl chloride, sulfuryl chloride, acetyl chloride, phosphoryl chloride and oxalyl bromide, thionyl bromide, sulfuryl bromide, acetyl bromide, phosphoryl bromide. Preference is given to the chlorinating agents. Preference is given to oxalyl chloride. The halogenating agent relative to the acid is preferably used in a molar equivalent ratio of from 1:1 to 5:1, preferably about 1.08 to 1.

The solvents used to perform the reaction, both the halogenation reaction and the ester formation, may be all organic inert solvents. Preference is given to polar organic solvents, for example ketones such as acetone, methyl ethyl ketone, methyl isobutyl ketone, ethers such as tetrahydrofuran (THF) or dioxane, chlorinated solvents such as dichloromethane, chloroform and related compounds, and polar aprotic solvents such as acetonitrile. Preference is given to methyl ethyl ketone, methyl isobutyl ketone, tetrahydrofuran, dioxane, and also polar aprotic solvents, preferably acetonitrile.

The temperature for the formation of the acid halide, preferably of the acid chloride, is preferably in the range from −10° C. to 50° C., preferably between 20° C. and 30° C.

The temperature for the coupling or ester formation is preferably between 20° C. and the reflux temperature of the solvent, preferably between 60° C. and 100° C.

The molar equivalent ratio of 3-indolecarboxylic acid or of the corresponding acid halide to the quinolizine compound is preferably from about 1:1 to 5:1, preferably about 1.3:1.

After the reaction or ester formation has ended, the ester formed can be released by neutralizing the acidic reaction mixture with a base, preferably with an inorganic base such as potassium carbonate, sodium carbonate, sodium hydrogen carbonate.

The present invention also relates to a method for precipitating and/or purifying dissolved crude dolasetron, which is characterized in that it is precipitated out of the solvent which is preferably selected from the group comprising polar organic solvents and/or polar aprotic solvents, preferably ketones, ethers, chlorinated solvents and/or aprotic solvents, by adding an apolar hydrocarbon compound having a polarity E^{o} in the region of <0.20, preferably <0.10 [or a dielectric constant (20° C.) of <5.0, preferably <3.0], preferably a saturated or unsaturated, linear, branched or cyclic hydrocarbon, preferably by adding hexane, heptane, petroleum ether and/or cyclohexane, preferably cyclohexane. The present invention also relates to a process for purifying crude dolasetron, which is characterized in that it is dissolved in a solvent selected from the group comprising polar organic solvents and polar aprotic solvents, preferably comprising ketones, ethers, chlorinated solvents and polar aprotic solvents, preferably in acetone or methyl ether ketone, and precipitated by adding a strong acid, by means of salt formation. The strong acid is preferably sulfuric acid (formation of the sulfate or hydrogensulfate), by addition of an organic sulfonic acid, preferably by addition of methanesulfonic acid (formation of the mesylate), of benzenesulfonic acid (formation of the besylate), of toluenesulfonic acid (formation of the tosylate), of trifluoromethanesulfonic acid (formation of the trifluoromethanesulfonate), or of camphorsulfonic acid (formation of the camphorsulfonic acid salt). Preference is given to precipitating the hydrogensulfate, the mesylate, the besylate, the tosylate or the trifluoromethanesulfonate, preferably the mesylate.

The salts of dolasetron obtained in this way, i.e. dolasetron sulfate, dolasetron hydrogensulfate, dolasetron besylate, dolasetron tosylate, dolasetron trifluoromethanesulfonate, dolasetron camphorsulfonate, are novel and are provided by the present invention.

Likewise novel are the salts mentioned of the quinolizine compound, i.e. of hexahydro-8-hydroxy-2,6-methano-2H-quinolizin-3(4H)-one, with a very strong acid. In this context, the salts mentioned of hexahydro-8-hydroxy-2,6-methano-2H-quinolizin-3(4H)-one, i.e. the sulfate, the hydrogensulfate, the mesylate, the besylate, the tosylate and the trifluoro-methanesulfonate, are also novel and are provided by the present invention.

The quinolizine compound, i.e. hexahydro-8-hydroxy-2,6-methano-2H-quinolizin-3(4H)-one, can be prepared according to the following scheme 2:

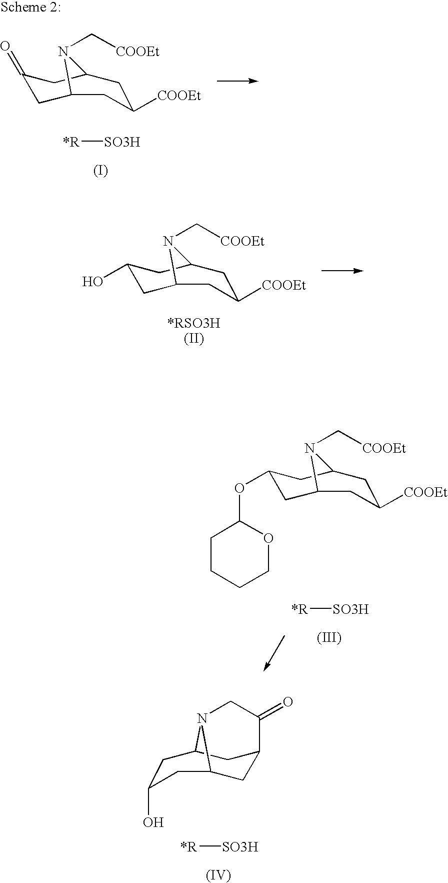

The name *R—SO₃H in the above scheme means that the compounds of the formulae (I), (II), (III) and (IV) may be present either as the free base or as the salt, preferably as the sulfate, hydrogensulfate, mesylate, besylate, tosylate, trifluoromethanesulfonate, or as the camphorsulfonate. The compounds of the formulae (I), (II) and (III) in the form of these salts are also novel and are provided by the present invention.

The compound of the formula (I) is prepared in a manner known per se, as shown in the following scheme 3:

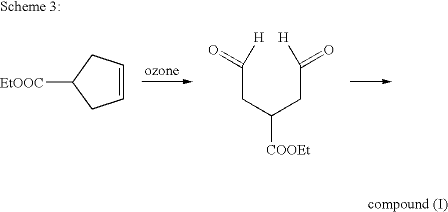

The reaction with ozone is known from B. E. Jacobson et al., Angewandte Chemie, International Edition (2002), 41 (16), 3059-61) and EP 0 339 669. Alternatively, dihydroxylation with osmium tetroxide and a subsequent periodate cleavage of the diol to the dialdehyde is described in EP 0 266 730, EP 0 329 902, EP 309 903, EP 0 329 904, EP 0 329 905, EP 0 330 788, EP 330 824, EP 0 339 669, and also U.S. Pat. No. 4,906,755 and U.S. Pat. No. 5,011,846. The conversion of the dialdehyde to the compound (I) is described in EP 0 266 730 and the other patent literature cited above.

The dialdehyde in the above formula scheme can also be obtained by opening the dihydropyran compound, as shown in the following scheme 4:

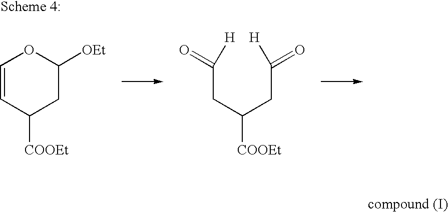

The process for converting the dihydropyran compound to the dialdehyde consists in treating the dihydropyran compound in aqueous or mixed aqueous solutions or emulsions with an acid, which opens the acetal to give the dialdehyde. Preference is given to the reaction of the dihydropyran pH<5, preferably pH<3, in the range from 20° C. up to the reflux temperature of the solvent, preferably in the range from 50° C. to 100° C., in the presence of a medium-strength to strong acid which is soluble in water and has a pKa of preferably <5, preferably <3, or an acid which is soluble in the solvent mixture used, preferably sulfuric acid, methanesulfonic acid, benzenesulfonic acid, toluene-sulfonic acid, trifluoromethanesulfonic acid or camphorsulfonic acid, preferably methanesulfonic acid and toluenesulfonic acid.

The examples which follow illustrate the invention.

### EXAMPLE 1

### Preparation of 7-ethoxycarbonyl-9-(ethoxy-carbonylmethyl)-9-azabicyclo[3.3.1]nonan-3-one methane-sulfonate; example of a compound of the formula (I)

41.29 g (200 mmol) of ethyl 2-ethoxy-3,4-dihydro-2H-pyrancarboxylate are mixed with 400 ml of water. 1.90 g (10 mmol) of p-toluenesulfonic acid are added to the resulting milky emulsion and the mixture is heated to 60-80° C. Once a clear solution has formed, the mixture is cooled to room temperature, and 20.90 g (120 mmol) of dipotassium hydrogenphosphate, 43.83 g (300 mmol) of acetone-1,3-dicarboxylic acid and 30.71 g (220 mmol) of glycine ethyl ester hydrochloride are added successively to the ethyl 4-oxo-2-(2-oxoethyl)-butanoate formed. After complete reaction, the mixture is acidified with 29.9 g (262 mmol) of 32% hydrochloric acid and extracted with tert-butyl methyl ether. The organic phase is discarded, and the aqueous phase is basified with 81.0 g (608 mmol) of 30% sodium hydroxide solution and extracted again with tert-butyl methyl ether. The organic phase is concentrated on a rotary evaporator, taken up in 64 g of acetone and admixed with 10.27 g (107 mmol) of methanesulfonic acid. After a continued stirring time of 2 hours, the crystals formed are filtered off, washed with a little cold acetone and dried to constant weight under reduced pressure; yield 38.56 (47%), colorless crystals.

### EXAMPLE 2

### Preparation of 7-ethoxycarbonyl-9-(ethoxy-carbonylmethyl)-9-azabicyclo[3.3.1]nonan-3-ol; example of a compound of the formula (II)

250 g (0.597 mmol) of 7-ethoxycarbonyl-9-(ethoxycarbonylmethyl)-9-azabicyclo[3.3.1]nonan-3-one methane-sulfonate are suspended in 650 g of absolute ethanol. The reaction mixture is neutralized by adding 196.7 g (0.607 mmol) of sodium methoxide (21% in ethanol). A solution of 37.83 g (0.599 mol) of sodium borohydride in a solution of 500 g of absolute ethanol and 2.5 g of sodium ethoxide (21% in ethanol) are then added at room temperature. After complete reduction of the keto group, 139.9 g of acetone are added in order to destroy the excess reagent. The mixture is neutralized by adding 84 g (0.738 mol) of 32% hydrochloric acid, and concentrated on a rotary evaporator. The aqueous residue is taken up in ethyl acetate and, after phase separation, the organic phase is washed with sodium chloride solution and water. Subsequently, the organic phase is concentrated to dryness, yield 186.5 g (87%) of viscous, light brown oil.

### EXAMPLE 3

### Preparation of 7-ethoxycarbonyl-9-(ethoxy-carbonylmethyl)-3-(2-tetrahydro-2H-pyranyloxy)-9-azabicyclo[3.3.1]nonane methanesulfonate; example of a compound of the formula (III)

176.10 g (content 85%, 0.50 mol) of 7-ethoxycarbonyl-9-(ethoxycarbonylmethyl)-9-azabicyclo[3.3.1]nonan-3-ol are dissolved in 433.0 g of 1,2-dimethoxyethane. 62.47 g (0.65 mol) of methanesulfonic acid and 75.91 g (0.90 mmol) of 3,4-dihydro-2H-pyran are added. A yellow suspension forms. After addition of 444 g of tert-butyl methyl ether, the mixture is filtered, and the filter-cake is washed with 74 g of tert-butyl methyl ether. The solid is dried to constant weight under reduced pressure; yield 233.5 g (97.4%) of beige solid.

### EXAMPLE 4

### Preparation of trans-hexahydro-8-hydroxy-2,6-methano-2H-quinolizin-3(4H)-one (crude); example of a compound of the formula (IV)

92.80 g (200 mmol) of 7-ethoxycarbonyl-9-(ethoxy-carbonylmethyl)-3-(2-tetrahydro-2H-pyranyloxy)-9-azabicyclo[3.3.1]nonane methanesulfonate are suspended in 178.0 g of tetrahydrofuran. 16.83 g (230 mmol) of tert-butylamine are added and the mixture is stirred for 3 hours. The mixture is filtered. The filtrate is added at room temperature to a suspension of 51.63 g (460 mmol) of potassium tert-butoxide in 107 g of tetrahydrofuran. The resulting dark solution is heated to reflux temperature. The solvent is distilled off as far as possible. 160 g of water are added to the residue. An orange solution is obtained. The remaining organic solvent is distilled off. The resulting brown-orange aqueous emulsion is extracted with 120 g of 2-butanone and, after phase separation, the organic phase is admixed with 17.30 g (180 mmol) of methane-sulfonic acid. The mixture is stirred under reflux for 3 h. After neutralization with a solution of 27.64 g (200 mmol) of potassium carbonate in 200 ml of water, the phases are separated. The organic phase is concentrated as far as possible on a rotary evaporator. 36.25 g (content approx. 75%) of a viscous brown oil are obtained.

### EXAMPLE 5

### Preparation of trans-hexahydro-8-hydroxy-2,6-methano-2H-quinolizin-3(4H)-one methylsulfonate; example of a compound of the formula (IV)

36.25 g (content 75%, 150 mmol) of crude trans-hexahydro-8-hydroxy-2,6-methano-2H-quinolizin-3(4H)-one are dissolved in a mixture of 161 g of methyl ethyl ketone and 118 g of 2-propanol. This results in a brownish solution. 17.30 g (180 mmol) of methanesulfonic acid are added dropwise to this solution at 20-30° C. The solution is heated to reflux temperature (77-80° C.) and stirred at this temperature for 3 hours. Even in the course of heating, an ochre suspension forms. After the cooling, the mixture is stirred at 20-30° C., then filtered and washed with 64 g of methyl ethyl ketone, and the product is dried at 65° C. Yield 39.46 g (purity 96%, content-corrected yield based on the crude product 91%).

### EXAMPLE 6

### Preparation of trans-hexahydro-8-hydroxy-2,6-methano-2H-quinolizin-3(4H)-one toluenesulfonate

7.00 g (38.6 mmol) of trans-hexahydro-8-hydroxy-2,6-methano-2H-quinolizin-3(4H)-one and 7.35 g (38.6 mmol) of p-toluenesulfonic acid monohydrate are initially charged. 55 mol of ethanol are added and the mixture is stirred. The suspension is heated to reflux (internal temperature (IT)=78° C.), and 5.0 ml of water are added so as to obtain a clear solution at reflux. The solution is cooled to room temperature and stirred for 1-2 hours. The suspension is filtered and the white solid is washed with 10 ml of ethanol. The moist product is dried overnight at 55° C. in a vacuum drying cabinet. 9.54 g (70%) of white crystalline solid are obtained.

### EXAMPLE 7

### Preparation of trans-hexahydro-8-hydroxy-2,6-methano-2H-quinolizin-3(4H)-one (+)-camphor-10-sulfonate

7.00 g (38.6 mmol) of trans-hexahydro-8-hydroxy-2,6-methano-2H-quinolizin-3(4H)-one and 8.97 g (38.6 mmol) of (+)-camphor-10-sulfonic acid are initially charged. 70 mol of ethanol are added and the mixture is stirred. The suspension is heated to reflux (IT=78° C.) and 11.0 ml of water are added so as to obtain a clear solution at reflux. The solution is cooled to room temperature and stirred for 1-2 hours. The suspension is filtered and the white solid is washed with 10 ml of ethanol. The moist product is dried overnight at 55° C. in a vacuum drying cabinet. 9.29 g (58.2%) of white crystalline solid are obtained.

### EXAMPLE 8

### Preparation of trans-hexahydro-8-hydroxy-2,6-methano-2H-quinolizin-3(4H)-one trifluoromethane-sulfonate

7.00 g (38.6 mmol) of trans-hexahydro-8-hydroxy-2,6-methano-2H-quinolizin-3(4H)-one and 5.80 g (38.6 mmol) of trifluoromethanesulfonic acid are initially charged. 42 mol of isopropanol are added and the mixture is stirred. The suspension is heated to reflux (IT=80° C.) and a clear solution at reflux is obtained. The solution is cooled to 0-5° C. and stirred for 1-2 hours. The suspension is filtered and the white solid is washed with 10 ml of isopropanol. The moist product is dried overnight at 55° C. in a vacuum drying cabinet. 11.05 g (86.4%) of white crystalline solid are obtained.

### EXAMPLE 9

### Preparation of trans-hexahydro-8-hydroxy-2,6-methano-2H-quinolizin-3(4H)-one benzenesulfonate

7.00 g (38.6 mmol) of trans-hexahydro-8-hydroxy-2,6-methano-2H-quinolizin-3(4H)-one and 6.11 g (38.6 mmol) of benzenesulfonic acid are initially charged. 49 mol of ethanol are added and the mixture is stirred. The suspension is heated to reflux (IT=78° C.) and a clear solution at reflux is obtained. The solution is cooled to room temperature and stirred for 1-2 hours. The suspension is filtered and the white solid is washed with 10 ml of ethanol. The moist product is dried overnight at 55° C. in a vacuum drying cabinet. 8.29 g (63.3%) of white crystalline solid are obtained.

### EXAMPLE 10

### Preparation of trans-hexahydro-8-hydroxy-2,6-methano-2H-quinolizin-3(4H)-one sulfate

7.00 g (38.6 mmol) of trans-hexahydro-8-hydroxy-2,6-methano-2H-quinolizin-3(4H)-one and 4.00 g (38.6 mmol) of 95-97% of sulfuric acid are initially charged. 70 mol of isopropanol are added and the mixture is stirred. The suspension is heated to reflux (IT=80° C.) and 9.5 ml of water are added, so as to obtain a clear solution at reflux. The solution is cooled to 0-5° C. and stirred for 1-2 hours. The suspension is filtered and the white solid is washed with 10 ml of isopropanol. The moist product is dried overnight at 55° C. in a vacuum drying cabinet. 8.30 g (77%) of white crystalline solid are obtained.

### EXAMPLE 11

### Preparation of trans-hexahydro-8-(3-indolylcarbonyloxy)-2,6-methano-2H-quinolizin-3(4H)-one methylsulfonate monohydrate; dolasetron

112.51 g (400 mmol) of trans-hexahydro-8-hydroxy-2,6-methano-2H-quinolizin-3(4H)-one methylsulfonate and 83.80 g (520 mmol) of indolecarboxylic acid are suspended in 450 g of methyl ethyl ketone (2-butanone). Under a nitrogen protective gas atmosphere, 71.10 g (560 mmol) of oxalyl chloride are added within 60 minutes. Gas evolution is observed. The mixture heated to reflux temperature (76-78° C.) and stirred for 1.5 hours. After cooling to 20-30° C., 480 g of methyl ethyl ketone are added to the suspension, and then a solution of 138.21 g (1000 mmol) of potassium carbonate in 322.5 g of purified water is added. This results in a biphasic yellowish suspension. This is heated to a temperature of 70° C., in the course of which the solid goes completely into solution. The lower aqueous phase is removed, and the organic phase is washed once at 70° C. with 200 g of purified water. Subsequently, the mixture is cooled to 15-20° C. and 187 g of cyclohexane are added. The mixture is stirred for 2 hours, and the solid is filtered off and washed with 64 g of methyl isobutyl ketone. The solid can be processed further directly, for example to give the salt. When the solid is dried at 60° C., 116.57 g (85%) of a beige solid are obtained.

### EXAMPLE 12

### Preparation of trans-hexahydro-8-(3-indolylcarbonyloxy)-2,6-methano-2H-quinolizin-3(4H)-one methylsulfonate monohydrate; dolasetron methylsulfonate monohydrate

The solid obtained according to example 11 is admixed with 5.83 g of activated carbon and slurried addition of 935 g of acetone. The black suspension is heated to reflux temperature (56° C.). The mixture is then filtered through a suction filter preheated to 50° C. The filtercake is washed with 30.0 g of acetone. The combined clear, slightly yellowish filtrates are admixed with 35.0 g of purified water. 34.07 g (355 mmol) of methanesulfonic acid are added, and the mixture is heated to reflux (56° C.). After a continued stirring time of 5 minutes at reflux, the mixture is cooled to 10-15° C. and stirred at this temperature for 3 hours, and the resulting precipitate is filtered off. The filtercake is washed with 185 g of acetone and dried at IT 40-43° C. Yield: 139.39 g (79.5% of theory), purity 99.7%.

1. A process for preparing esters of optionally substituted 3-indolecarboxylic acid with hexahydro-8-hydroxy-2,6-methano-2H-quinolizin-3(4H)-one, by converting optionally substituted 3-indolecarboxylic acid with a suitable halogenating agent to the corresponding acid halide, preferably to the acid chloride, and reacting the latter with hexahydro-8-hydroxy-2,6-methano-2H-quinolizin-3(4H)-one, characterized in that the overall reaction is performed in an acidic medium at an acid value (pH) of not more than 7.

2. The process as claimed in claim 1, characterized in that unsubstituted 3-indolecarboxylic acid is converted with a suitable halogenating agent to the corresponding acid chloride and the latter is reacted with either hexahydro-8-hydroxy-2,6-methano-2H-quinolizin-3(4H)-one or endo-hexahydro-8-hydroxy-2,6-methano-2H-quinolizin-3(4H)-one.

3. The process as claimed in claim 1, characterized in that the overall reaction is performed at a pH of at most 6.5.

4. Process as claimed in claim 1, characterized in that the acidic pH in the reaction mixture is established by using a strong inorganic acid, preferably sulfuric acid, and/or a strong organic acid, preferably methanesulfonic acid, benzenesulfonic acid, toluenesulfonic acid, trifluoromethanesulfonic acid and/or camphorsulfonic acid, preferably sulfuric acid, methanesulfonic acid, benzenesulfonic acid, toluenesulfonic acid and/or trifluoromethanesulfonic acid, preferably methanesulfonic acid.

5. The process as claimed in claim 1, characterized in that the 3-indolecarboxylic acid is converted in acidic medium to the corresponding acid halide and then the quinolizine compound is added to the reaction mixture.

6. The process as claimed in claim 1, characterized in that the 3-indolecarboxylic acid is initially charged in acidic medium together with the quinolizine compound, and the corresponding acid halide is prepared in situ by adding a suitable halogenating agent and is then reacted further in the reaction mixture with the quinolizine compound.

7. The process as claimed in claim 1, characterized in that a salt of the quinolizine compound with a strong acid is prepared beforehand, preferably a salt of the endo-alcohol, preferably the salt formed with sulfuric acid, preferably the hydrogensulfate, or the salt of an organic sulfonic acid, preferably the mesylate, the besylate, the tosylate, the trifluoromethanesulfonate, or the camphorsulfonate, preferably the hydrogensulfonate, the mesylate, the besylate, the tosylate or the trifluoromethanesulfonate, preferably the mesylate, and this salt is used in the reaction.

8. The process as claimed in claim 1, characterized in that a salt of 3-indolecarboxylic acid with a very strong acid is prepared beforehand, preferably the salt formed with sulfuric acid, preferably the hydrogensulfate, and this salt is used in the reaction.

9. The process as claimed in claim 1, characterized in that the solvent used to perform the reaction is an organic inert solvent, preferably a polar organic solvent; preferably a ketone, preferably acetone, methyl ethyl ketone, methyl isobutyl ketone; or an ether, preferably tetrahydrofuran or dioxane; a chlorinated solvent, preferably dichloromethane, chloroform and related compounds; and/or a polar aprotic solvent, preferably acetonitrile.

10. The process as claimed in claim 1, characterized in that the formation of the acid halide, preferably of the acid chloride, is performed at a temperature in the range from −10° C. to 50° C., preferably between 20° C. and 30° C., and ester formation at a temperature in the range of 20° C. and the reflux temperature of the solvent, preferably between 60° C. and 100° C.

11. The process as claimed in claim 1, characterized in that the molar equivalent ratio of 3-indolecarboxylic acid or the corresponding acid halide to the quinolizine compound is in the range from 1:1 to 5:1.

12. A process for precipitating and/or purifying dissolved crude dolasetron, characterized in that it is precipitated out of the solvent, preferably selected from the group comprising polar organic solvents and/or polar aprotic solvents, preferably ketones, ethers, chlorinated solvents and/or aprotic solvents, by adding an apolar hydrocarbon compound having a polarity E^{o} in the region of <0.20, preferably <0.10, preferably by adding a saturated or unsaturated, linear, branched or/or cyclic hydrocarbon, preferably by adding hexane, heptane, petroleum ether and/or cyclohexane, preferably cyclohexane.

13. A process for purifying crude dolasetron, characterized in that it is dissolved in a solvent selected from the group comprising polar organic solvents and polar aprotic solvents, preferably comprising ketones, ethers, chlorinated solvents and polar aprotic solvents, preferably in acetone or methyl ether ketone, and precipitated by adding a strong acid, preferably sulfuric acid or an organic sulfonic acid, by means of salt formation, preferably by adding methanesulfonic acid, benzenesulfonic acid, toluenesulfonic acid, trifluoromethanesulfonic acid or camphorsulfonic acid.

14. The compounds dolasetron sulfate, dolasetron hydrogensulfate, dolasetron besylate, dolasetron tosylate, dolasetron trifluoromethanesulfonate, dolasetron camphorsulfonate.

15. The compounds: hexahydro-8-hydroxy-2,6-methano-2H-quinolizin-3(4H)-one sulfate, hexahydro-8-hydroxy-2,6-methano-2H-quinolizin-3(4H)-one hydrogensulfate, hexahydro-8-hydroxy-2,6-methano-2H-quinolizin-3(4H)-one mesylate, hexahydro-8-hydroxy-2,6-methano-2H-quinolizin-3(4H)-one besylate, hexahydro-8-hydroxy-2,6-methano-2H-quinolizin-3(4H)-one tosylate, hexahydro-8-hydroxy-2,6-methano-2H-quinolizin-3(4H)-one trifluoromethanesulfonate.

16. A process for preparing hexahydro-8-hydroxy-2,6-methano-2H-quinolizin-3(4H)-one, characterized in that the procedure is according to the following scheme 2 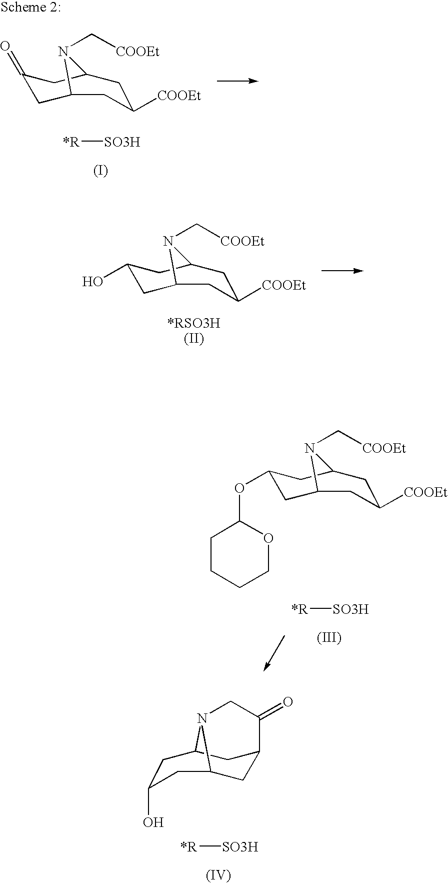
where the name *R—SO₃H means that the compounds of the formulae (I), (II), (III) and (IV) may be present either as a free base or as a salt, preferably as the sulfate, hydrogensulfate, mesylate, besylate, tosylate, trifluoromethanesulfonate, or as the camphorsulfonate.

17. The compounds of the formulae (I), (II) and (III) according to claim 16 in the form or the salts mentioned in claim 16.

18. A process for preparing the compound of the formula (I) as claimed in claim 16, characterized in that the dialdehyde is obtained according to the following scheme 4 by opening the dihydropyran compound according to scheme 4: 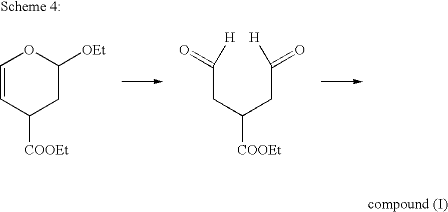
by treating the dihydropyran compound in aqueous or mixed aqueous solution or as emulsions with an acid to open the acetal to give the dialdehyde.

19. The process as claimed in claim 18, characterized in that the reaction of the dihydropyran is performed at a pH of pH<5, preferably at pH<3, in the range from 20° C. up to the reflux temperature of the solvent, preferably in the range from 50° C. to 100° C., in the presence of a medium-strength to strong, water-soluble acid having a pKa of preferably <5, preferably <3, or acid soluble in the solvent mixture used, preferably sulfuric acid, methanesulfonic acid, benzenesulfonic acid, toluenesulfonic acid, trifluoromethanesulfonic acid or camphorsulfonic acid, preferably methanesulfonic acid and toluenesulfonic acid.
